(19) United States
(12) Patent Application Publication
(10) Pub. No.: US 2008/0149501 A1
(43) Pub. Date: Jun. 26, 2008

(54) Set Comprising Ion-Selective Solid-Contact Electrodes

(75) Inventors: Martin Heule, Birmensdorf (CH); Erno Pretsch, Uetikon am See (CH); Tamas Vigassy, Zurich (CH)

Correspondence Address: SHOEMAKER AND MATTARE, LTD, 10 POST OFFICE ROAD - SUITE 110, SILVER SPRING, MD, 20910, US

(73) Assignee: METROGLAS AG, Affoltern a/A. (CH)

(21) Appl. No.: 11/962,422

(22) Filed: Dec. 21, 2007

(30) Foreign Application Priority Data
Dec. 22, 2006 (EP) 06127154.0

(51) Int. Cl. G01N 27/26; B05D 5/12; A61L 2/00

(52) U.S. Cl. 205/788.5; 204/416; 204/418; 427/77; 205/775; 422/1

(57) ABSTRACT

A set (1) comprising at least two ion-selective solid-contact electrodes comprises at least two ion-selective measuring points (6). Each measuring point (6) comprises a metal conductor (3) and/or an inner layer (7) of a conductive polymer and an outer, ion-selective membrane (8). At least two measuring points (6) are selective in each case with respect to a different ion of an analysis solution. One of the solid-contact electrodes can be used as a reference electrode.

The invention relates to a set comprising ion-selective solid-contact electrodes, a method for the production of such an electrode set, methods for the quantitative analysis of ions in solutions, the use of a solid-contact electrode as a reference electrode and a method for the preparation of such electrodes for measurement, having the features according to the independent claims.

Polymer membrane ion-selective electrodes (ISE) have been used for decades. The ion selectivity of such ISE is determined by the choice of suitable membrane components. Usually, these comprise selective ion complexing agents (ionophors) and a lipophilic counter, ion, such as, for example, phenylborates. The resulting ion-dependent potential according to the Nernst equation is measured by means of potentiometry. The membrane polymer is usually prepared from a mixture of polyvinyl chloride and a plasticizer. Mixtures of methacrylate polymers, silicones or polyurethanes are also known.

US 20030217920 describes a plasticizer-free ISE comprising a methacrylate copolymer. Regarding the known ISE and the production thereof, reference is also made to Analytica Chimica Acta 2001, 443, 25 (L. Y. Heng, E. A. H. Hall), the content of which is hereby incorporated in this application. Further details in this context are also described in Ion-Sel. Electrode Rev. 1988, 10, 71 (G. J. Moody, B. B. Saad, J. D. R. Thomas).

Conventionally ion-selective electrodes are used in combination with a silver/silver chloride electrode. In the past, considerable attempts were made to replace the internal liquid discharge required for this purpose by contacts comprising thin solid-state layers. The advantages lie in the simplified design, for the production of which known coating or thin-film technologies or printing processes are used. These permit economical production. These techniques also permit miniaturization of the ISE and the production of disposal ISEs.

EP 1 480 038 A1 describes an ISE produced by means of screen printing and having an internal electrode comprising a conductor paste. The paste is a mixture of a water-soluble salt and an alkaline earth metal.

JP 2002 039990 describes an ion-selective electrode having a silver layer, a silver halide layer, an electrolyte layer and an ion-selective film.

It was possible to show that water can diffuse through an ISE membrane and can be deposited on the internal electrode, which results in a shift of the measured potential. The use of hydrophobic conductive polymers, such as, for example, polythiophenes or polypyrroles, as internal electrodes is also known. These can prevent the deposition of water and result in a more stable behaviour of the ISE (cf. for example Electroanalysis 18 (1), 2006, 7 to 18, in particular 12, J. Bobacka).

T. Blaz et al. (cf. Analyst, 130, 2005, 637 to 643) propose the use of reference electrodes comprising a conductive polymer which is provided with a pH buffer system. However, this approach is specific to the use of a sample solution within a certain pH range and therefore not suitable for a broad field of use. In addition interference by redox-active substances imposes limits to the use of such electrodes in practice. Moreover, these polymer films must be conditioned before use.

The membrane potential must be measured relative to a reference electrode having a stable electrochemical potential. Usually, this is effected via a silver/silver chloride electrode. However, the flattening and miniaturization of stable silver/silver chloride reference electrodes which are easy to handle is a problem which is unsolved to date and prevents broad use of solid-contact ISE.

US 2001032785 describes a method for the production of flat reference electrodes. The main problems here lies in keeping the chloride activity at the silver/silver chloride layer constant. Small reservoirs of chloride are rapidly exhausted and lead to a shift in the reference potential. Such reference electrodes are therefore greatly limited in their life and are suitable only for a limited number of measurements. In addition, there is the problem that the connections are not sufficiently durable, so that silver ions come into contact with the sample solution. This may lead to undesired precipitations of sliver ions and proteins which are present in the sample matrix. Moreover, this leads to a shift in the reference potential or to blocking of the membrane. These problems are solved in the case of a conventional standard reference electrode by liquid connections via porous ceramic layers or a fixed coating of the connections.

There are already so-called titrodes which are composed of a pH glass membrane and a metal electrode. However, the pH glass membrane requires careful shielding, so that planarization and miniaturization are scarcely realizable.

It is therefore an object of the present invention to avoid the disadvantages of the known electrode, in particular to provide an apparatus of the type mentioned at the outset and a method for the production thereof; this apparatus should be capable of being produced in a simple manner and of being used in a flexible manner. In particular, the apparatus should be capable of being economically produced and miniaturized.

According to the invention these objects are achieved by a set comprising at least two ion-selective solid-contact electrodes and a method for the production thereof having the features of the independent claims.

The set according to the invention has at least two ion-selective solid-contact electrodes. These each comprise at least one ion-selective measuring point, preferably only one ion-selective measuring point. At least two of the measuring points are selective with respect to different ions of an analysis solution. With this set arrangement, the conventional reference electrode can be dispensed with in a surprisingly simple manner if the activity of a reference ion is kept constant or predictable. The disadvantages of known reference electrodes are therefore also eliminated.

In the context of this application, the terms used, in particular ion-selective electrodes, have the meaning according to the definition in Joseph Wang, Analytical Electrochemistry, Wiley-VCH, New York, Third Ed., 2006.

The ion-selective solid-contact electrodes can, but need not, form a structural unit. It is conceivable to provide a collection of a multiplicity of individual ion-selective solid-contact electrodes, each on a separate support. Each ion-selective solid-contact electrode has a measuring point which is selective with respect to an ion. The collection thus comprises, for example, a multiplicity of electrodes, each of which is selective with respect to a specific ion. Depending on the ion to be analyzed, at least two electrodes can be chosen from the collection in order to form a specific set for the relevant analysis.

The measuring points preferably each comprise, preferably consist of, an inner layer of an electrically conductive polymer and an outer ion-selective membrane. Here, outer membrane is to be understood as meaning the membrane which can be brought directly into contact with an analysis solution during use as intended. Here, inner layer is understood as meaning a layer separated by the outer membrane from the analysis solution.

The outer membrane is particularly preferably plasticizer-free.

Each of the measuring points can be applied to a metal conductor, such as, for example, gold. The inner layer thus lies between the membrane and the metal conductor. The metal conductors serve for transmitting the potentials of the measuring points to measuring contacts. Of course, the metal conductors must be insulated from the analysis solution by an insulator. In a particularly advantageous embodiment, the measuring points, which are each selective with respect to different ions in an analysis solution, are fastened or can be fastened to a common electrically insulating support, such as, for example, a ceramic or a polymer support. Thus, the set comprising at least two ion-selective solid-contact electrodes preferably forms a structural unit, which permits a compact design. Preferably, the metal conductors with the measuring points rest on the common electrically nonconductive support. The support preferably has a plurality of measuring points, in particular 2 to 10, particularly preferably 2 to 4, which are selective with respect to different ions. Such a set on a common support can be used universally for the analysis of a very wide range of solutions, which greatly simplifies laboratory use and reduces the danger of confusion in the choice of electrodes.

The use of a metal conductor is not absolutely essential; instead, the inner layer can also perform the function of the metal conductor.

With a set comprising at least two ion-selective solid-contact electrodes, the problems of a liquid internal discharge can be avoided. There is therefore no necessity for a separate reference electrolyte in a reference electrode. Preferably, each measuring point has basically the same structure with the same components, and at least two outer membranes differ only through different ion-selective additives, in particular through different ionophors and/or different ion exchange salts. In the electrode arrangement according to the invention, each measuring point has virtually the same electrical resistance. As a result, said electrode arrangement has an electrically symmetrical structure. By the use of thin layers, the electrical resistance is comparatively low, typically 1-10 MOhm, which dispenses with the need for shielding the measuring points.

A further advantage of the apparatus according to the invention is that no reference electrode, in particular no Ag/AgCl reference electrode with a reference electrolyte, is necessary. With the electrode set according to the invention, there is no need to determine which measuring point gives the indicator and which measuring point gives the reference potential before measurement. This choice is preferably made by the analysis of the chemical composition of the analysis solution and additives thereof which are used for the analysis process.

The arrangement with a plurality of measuring points is particularly advantageous since the measuring points can be adapted in a simple manner to a multiplicity of ion analysis applications, such as, for example, titration or standard addition. Either an ion which is not the indicator ion to be investigated is already present in the analysis solution or said ion is added before or during the measuring of the analysis solution. The activity of this ion can then be used as reference activity during the entire quantitative determination.

Preferably, two or more measuring points of a set have the same ion selectivity. In this case, the set must have at least one further, but differently ion-selective, measuring point. This redundancy can increase the accuracy and reliability of the measurement and permits improved quality assurance.

For standardized laboratory analyses, disposable sensors are too expensive and the constant replacement thereof after each measurement is too complicated since to date no automatic changing apparatuses for the replacement are available. The electrode set according to the invention has a much longer life than conventional disposable sensors. It can therefore be used for a plurality of measuring cycles before it has to be replaced. In addition, the disadvantage of the reference electrode which quickly becomes unstable is absent in the invention.

Owing to the high prices for standard ISE, these are usually used until their response behaviour, in particular their slope, significantly deteriorates. This deterioration can assume such an extent that the quantitative results obtained with such an overused electrode are unreliable. The electrode set according to the invention can be replaced more often than conventional ISE but nevertheless has a much longer life than disposable sensors, so that it has to be replaced much later than disposable sensors. A typical life cycle of an electrode set according to the invention is preferably up to a few 100 analysis cycles with constant quality of the results of the measurement.

The inner layer of a measuring point preferably comprises a hydrophobic polymer, in particular a polythiophene, poly-(n-octyl)thiophene (POT), polydodecylthiophene (PDT), poly(2,2′-bithiophene), poly(3,4-ethylenedioxythiophene) (PEDOT), polyaniline or polypyrrole. These polymers form a particularly stable transition to the metal conductor.

The ion-selective membrane preferably comprises a polyvinyl chloride (PVC), an acrylate polymer or a methacrylate polymer (MA) or other polymers which contain an ion exchange salt and/or an ionophor.

Preferably, one of the measuring points is connected or can be connected as a reference sensor to a measuring input of a potentiometer. The connection is preferably effected via a contact at a free end of the metal conductor. At least one further measuring point gives the indicator signal via another contact.

In a further aspect, the electrode set according to the invention comprises a membrane which has a structure which is crosslinked via covalent bonds and/or is plasticizer-free.

This structure is preferably crosslinked by incorporated difunctional (meth)acrylate monomers.

The outer membrane are preferably obtainable from a mixture of a methacrylate, preferably 2-ethylhexyl methacrylate; a dimethacrylate, preferably 1,6-hexanediol dimethacrylate; a free radical initiator, such as, for example, 2,2′-azobis(2-methyl-butyronitrile) (AIBN) and an ion-specific component. The proportion of 1,6-hexanediol dimethacrylate is preferably about 0.1 to 5.0% by weight and the proportion of AIBN about 0.1 to 5.0% by weight.

These compositions are particularly advantageous because the respective identical base mixtures for outer membrane and inner layer can be used for all measuring points. Only the ion-specific component in the membrane varies at at least two measuring points. The symmetrical structure between two measuring points permits economical production of tailor-made membranes or electrodes with the use of the same reaction mixtures for the membranes and the production fluids. Based on the requirements of the users, an application-specific production process can be implemented.

Another aspect according to the invention relates to a method for the production of a set comprising at least two ion-selective solid-contact electrodes. For this purpose, at least two inner layers comprising an electrically conductive polymer, preferably POT, PDT or PEDOT, are applied to a support, which, for example, comprises ceramic. For example, the screen printing or the inkjet printing process is particularly suitable for this purpose. Alternatively, the inner layer can be produced directly on the electrode by electropolymerization of thiophene monomers. Some typical procedures are described in J. D. Guo, S. Amemiya, Anal. Chem., 2006, 78 (19), 6893-6902. Alternatively, for each electrode, a metal conductor can be applied between the inner layers and the support. The metal conductors are preferably printed onto the support. For each electrode, an ion-selective membrane is then applied, preferably printed onto the inner layers. A process particularly suitable for this purpose is inkjet printing. This method according to the invention is particularly suitable for the production of an electrode set according to the invention having at least two measuring points which are selective with respect to different ions.

This method is particularly advantageous because no reference electrode has to be produced in a separate and fundamentally different production method, as in the case of conventional electrodes. In particular, the coating of a substrate with silver/silver chloride is dispensed with.

After application, the layers can be cured, for example by heating. Alternatively or additionally, also with an appropriately chosen chemical composition, photopolymerization is suitable for this purpose.

The layers and/or the ion-selective membranes are preferably polymerized in situ; the reactants for the production of the polymer are thus printed on and not reacted until they are present on the support. Alternatively, the inner layer can be produced in situ by electropolymerization. This permits the use of printing or pipetting processes and simplifies the production method.

A further aspect of the invention comprises a method for measuring the ion activity in an analysis solution. For this purpose, a set comprising at least two ion-selective solid-contact electrodes with at least two measuring points which are each selective with respect to different ions of an analysis solution is immersed in the analysis solution. The potentials are then measured between two different measuring points and evaluated. The two measuring points are selective with respect to different ions. Preferably, a set comprising ion-selective solid-contact electrodes according to one of the embodiments described above is used for this purpose.

Preferably, a set of two ion-selective solid-contact electrodes, the chemical compositions of the measuring points of which differ only through different ionophors and/or ion exchange salts in the outer membranes thereof, is used for this method. This permits simple, economical production and allows an electrically symmetrical set arrangement.

The analysis of the ion activity with the electrode set described is used in standard addition processes or in titration.

The method for the analysis of the ion activity is preferably used for determining the equivalence point of the titration. The equivalence point can be determined, for example, by registration of a sudden change in the potential between two measuring points. The advantage is that it is not absolutely essential for an s-shaped titration curve to be present. A peak or a change in the slope of the curve is sufficient for determining the equivalence point. One use example is the determination of water hardness.

In the titration, the other ion which is not the ion to be analyzed is preferably either already present in the analysis solution or a solution having a suitable ion composition is mixed with the analysis solution before the titration. One measuring point is thus selective with respect to the ion to be analyzed, and another measuring point reacts selectively to an ion whose activity remains constant during the titration or whose changes of activity are predictable during the titration.

In each case, the potential difference between the two measuring points shows a distinguishable change at the equivalence point, so that the equivalence point can be determined using a suitable algorithm from the potentiometric measurement.

One aspect according to the invention also comprises a method for the determination of a reference electrode with a specified potentiometric analysis of a suitable analysis solution. For this purpose, the chemical composition of the sample to be analyzed is used: an ion which is present in the sample or is added to the sample before the analysis and the activity of which does not change or changes in a predictable manner during the analysis is defined. Thereafter, an ion-selective electrode, preferably an ion-selective solid-contact electrode, or a measuring point of an electrode set, which is selective with respect to the defined ion, is chosen. This electrode or the measuring point is then used as a reference electrode for the analysis. Thus, the choice of the reference electrode is determined not in the conventional manner by the sensor, which is taken as a reference electrode, but by the composition of the sample. An electrode set according to the invention is preferably used for this method.

In a further aspect of the invention, an electrode set according to the invention is installed in a laboratory apparatus. One of the ion-selective solid-contact electrodes is provided as the reference electrode. According to the invention, an ion-selective solid-contact electrode can also be used outside a laboratory apparatus as a reference electrode in an analysis process, such as, for example, titration or standard addition.

Completely surprisingly, it has additionally been found that a preferably plasticizer-free ion-selective solid-contact electrode can be prepared, in particular purified and/or sterilized, using heat and in particular under pressure without adversely affecting the outer membrane or inner layer and hence their function. For this purpose, the electrode or an electrode set according to the invention can be sterilized in a customary autoclave. Preferably, the electrodes are sterilized in the autoclave at a minimum temperature of 100° C., in particular at a minimum pressure of 1-2 bar. Preferably, the electrodes are sterilized in the course of at least 30 minutes. By means of this method according to the invention, the ion-selective solid-contact electrodes can thus be each sterilized in a simple manner prior to analysis without adversely affecting the function thereof. This permits the use of the electrodes several times, which reduces the costs of analyses.

Further individual features and advantages of the invention are evident from the following description of the working examples and from the drawings.

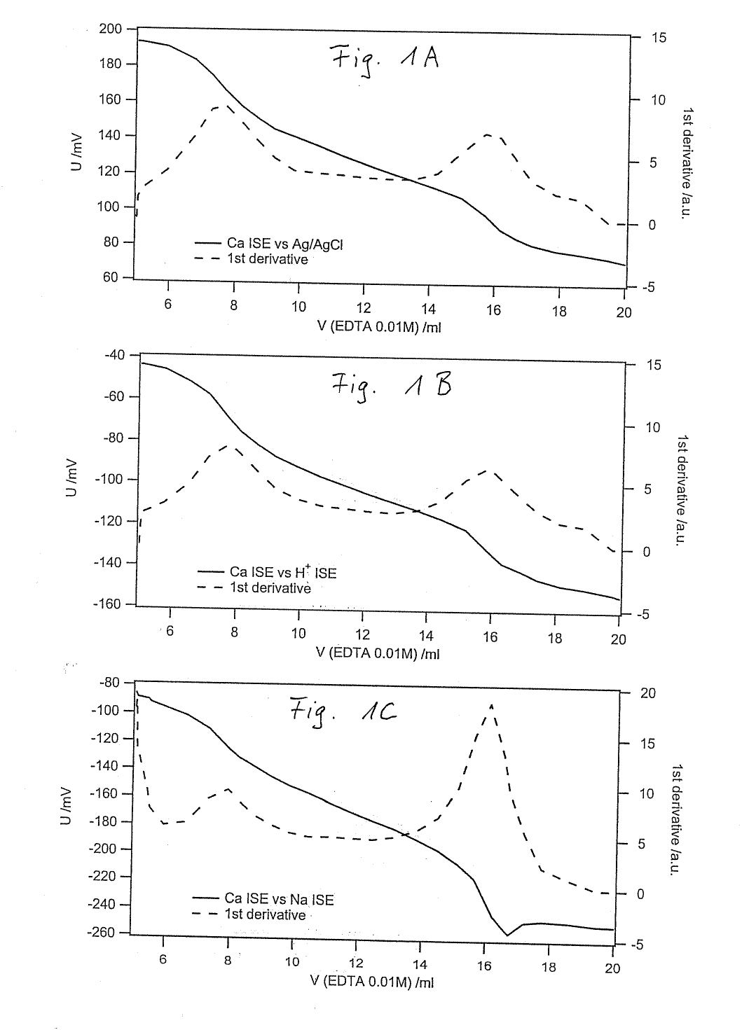

FIG. 1A shows a water hardness titration with the use of a Ca²⁺-ISE, referenced with a standard silver/silver chloride reference electrode,

FIG. 1B shows a water hardness titration with the use of a Ca²⁺-ISE and an H⁺-ISE as reference electrode,

FIG. 1C shows a water hardness titration using a Ca²⁺-ISE which is referenced relative to an Na⁺-ISE,

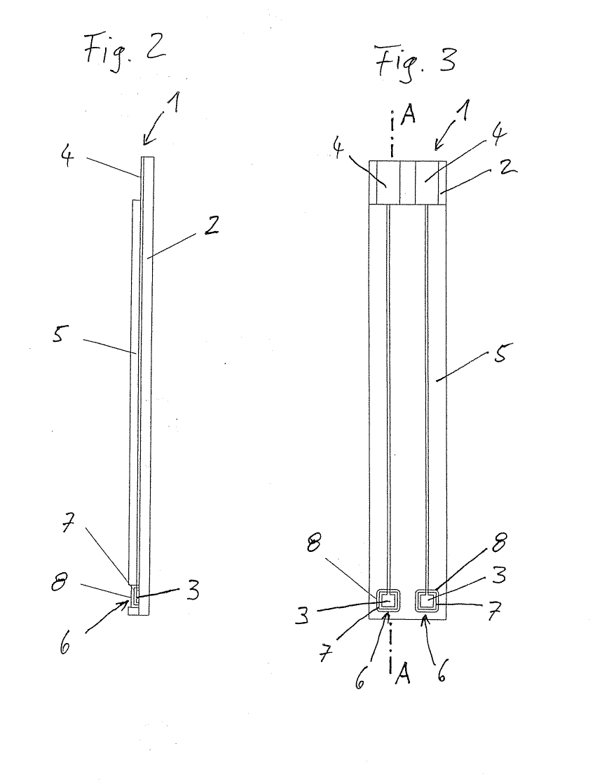

FIG. 2 shows a cross section of a first working example of an electrode set according to the invention along the sectional plane A-A according to FIG. 3,

FIG. 3 shows a view of a first working example of an electrode set according to the invention,

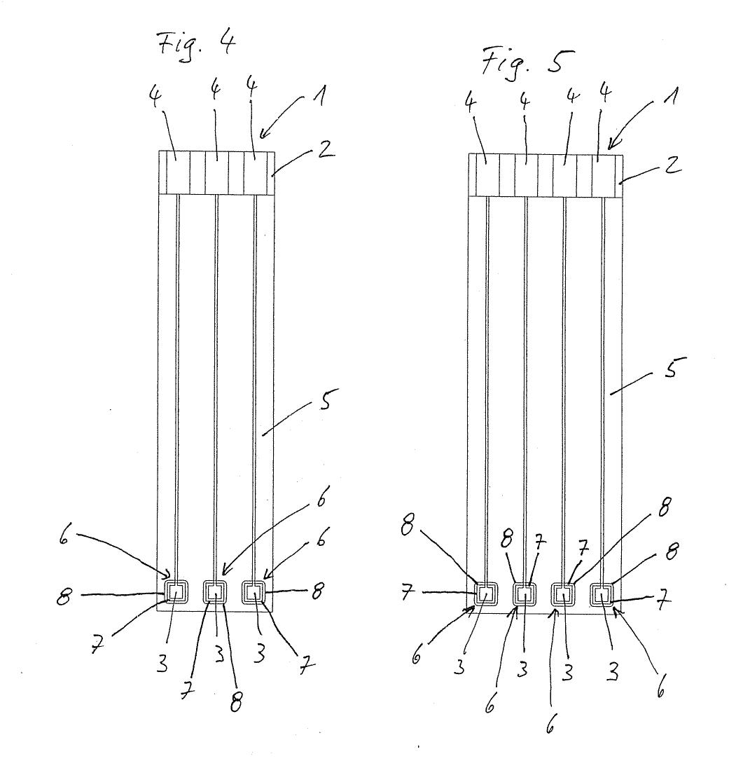

FIG. 4 shows a view of a second working example of an electrode set according to the invention,

FIG. 5 shows a view of a third working example of an electrode set according to the invention,

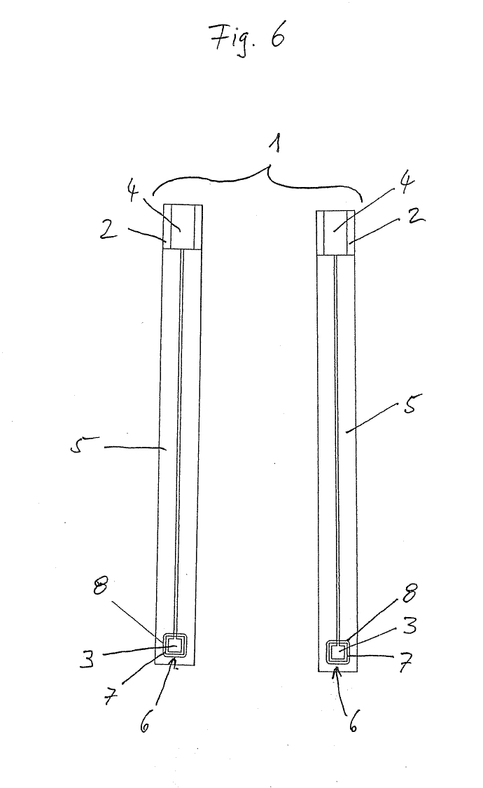

FIG. 6 shows a view of a fourth working example of an electrode set according to the invention.

FIGS. 1A to 1C show the varied applicability of the apparatus according to the invention for determining the water hardness by the determination of the Ca²⁺ and Mg²⁺ ions by complexometric titration in a water sample. Ethylenediaminetetraacetate (EDTA) was used as the titrant (complexing agent). 15 ml of a TRIS/acetylacetonate buffer solution were added to 150 ml of water sample before the titration. This buffer prevents interactions of trivalent cations and stabilizes the pH in order to obtain a constant complex formation rate of Ca²⁺ and Mg²⁺ with EDTA. The pH is therefore suitable as a reference potential which is tapped with a pH-ISE. In FIG. 1A, the water hardness is referenced via a Ca²⁺-ISE with a standard Ag/AgCl electrode. The same titration result is obtained with a Ca²⁺-ISE, referenced with an H⁺-ISE (FIG. 1B) or with an Na⁺-ISE (FIG. 1C), without changing the titration chemistry. Only the absolute values of the potential (ordinate) vary as a function of electrodes used and there is no need to add a further ion.

FIG. 2 shows a set (1) according to the invention comprising ion-selective solid-contact electrodes in cross section along the sectional plane A-A in FIG. 3. Two gold metal conductors 3 are applied to a ceramic support 2 by means of a screen printing process. Said metal conductors are parallel to one another and have, at one end, a broadened contact section 4 for tapping the potential. A broadened section is likewise present at the other end of each conductor 3. The support 2 and the two conductors 3 are provided with an insulating layer 5 comprising a nonconductive material. The insulating layer 5 is absent on the contact section 4 and on the opposite broadened section of the conductor 3, which belongs to a measuring point 6. The conductor 3 is provided at the measuring points 6 with an electrically conductive polymer layer 7. This is a poly-3-octylthiophene. The polymer of the layer 7 can be applied to the measuring point 6 for example from a solution by means of the drop casting process or inkjet printing; however, electrochemical coating is also conceivable. This polymer is optionally cured at a temperature between 40° C. and 180° C.

The inner layer is covered completely by an ion-selective membrane 8. The membrane 8 is a mixture of a 2-ethylhexyl methacrylate, 0.1 to 5.0% by weight of 1,6-hexanediol dimethacrylate and 1.0% by weight of 2,2′-azobis(2-methylpropionitrile). This mixture forms a crosslinked methacrylate polymer and forms a membrane matrix in which the ion-specific components are dissolved. For example, the ion-specific components for a Ca²⁺-ISE comprise 1.2% by weight of calcium ionophor IV (ETH 5234, N,N-dicyclohexyl-N′,N′-dioctadecyl-3-oxapentanamide) and 0.4% by weight of sodium tetrakis(3,5-bis(trifluoromethyl)phenyl)borate. These components are added to the base membrane mixture (see above). For other selective membranes, other ionophor molecules or ion exchange salts are mixed with the base membrane mixture, depending on the desired ion selectivity. The composition and the mixing ratio thereof are known to the person skilled in the art and/or can easily be determined in routine experiments.

A small amount of this ion-specific membrane mixture 8 is applied to the electrically conductive layer 7 so as to cover the entire area. As a result, no analysis solution can come into contact with the layer 7. The membrane is produced either by the inkjet printing process or by other pipetting processes; a so-called membrane cocktail is applied thereby and cures to give the membrane after application. The thickness of the membrane 8 is controlled via the applied volume of the membrane mixture 8 at the measuring point. Typically, the volume is 0.2 to 4 microlitres per measuring point.

As soon as the membrane mixture has been applied to the measuring point 6, the gel polymerization is started by activating the AIBN free radical initiator. Other free radical initiators, such as benzoin methyl ether, can be used in order to be able to reduce in a targeted manner the UV light power required for the polymerization. The activation takes place via heat and/or light. For each measuring point 6, a different methacrylate membrane mixture 8 is applied, and it is possible to use the same inkjet printing process or pipetting process in each case.

FIG. 3 shows a view of a set 1 according to the invention comprising two measuring points 6. The support 2 is printed with two parallel gold conductors 3. These have a contact section 4 at each end. This serves for tapping the potential from the measuring points 6 at the other end of the conductor 3. Each measuring point 6 comprises an inner layer 7 of a conductive polymer, wherein each layer rests on a conductor 3. Each of the inner layers 7 is covered by an ion-selective membrane 8, the ion-selective membranes 8 being formed so as to be ion-selective with respect to different ions.

The working examples shown in FIGS. 4 and 5 have a structure analogous to that of the sets 1 shown in FIGS. 2 and 3 and having ion-selective solid-contact electrodes. The set 1 shown in FIG. 4, however, has three measuring points 6. The set 1 shown in FIG. 5 has four measuring points 6. In order to enable a measuring point 6 to be used as a reference, in each case at least two of the four (FIG. 5) or 3 (FIG. 4) measuring points 6 are ion-selective with respect to different ions.

The example of an electrode set 1 in FIG. 6 is comparable with the working example shown in FIGS. 2 and 3. However, the measuring points 6 are arranged on separate supports 2 in FIG. 6. The two measuring points 6 of this electrode set 1 differ through a different ion selectivity. Which of the two measuring points 6 serves as a reference is not determined a priori by the electrode set but arises as a result of the analysis solution. In principle, both measuring points can be used as reference electrode or as indicator electrode.

1. Set, comprising at least two ion-selective solid-contact electrodes, each comprising an ion-selective measuring point, at least two of the measuring points being selective with respect to different ions.

2. Set according to claim 1, wherein each of the at least two measuring points comprises the following components:
an inner layer of an electrically conductive polymer;
an outer ion-selective membrane.

3. Set according to claim 1, wherein the at least two measuring points are each applied to a metal conductor.

4. Set according to claim 2, wherein each inner layer comprises a hydrophobic polymer.

5. Set according to claim 2, wherein the membrane comprises a substance selected from the group consisting of polyvinyl chloride (PVC), (meth)acrylate polymer (MA), polymers containing an ion exchange salt, polymers containing an ionophor.

6. Set according to claim 1, wherein two arbitrary measuring points are connected or can be connected to a measuring input of a potentiometer.

7. Set according to claim 2, wherein the membrane has a structure which is crosslinked via covalent bonds.

8. Set according to claim 7, wherein the structure is crosslinked by incorporated difunctional (meth)acrylate monomers.

9. Set according to claim 2, wherein the membrane is obtainable by reaction of the components mono(meth)acrylate, di(meth)acrylate and free radical initiator; and wherein the membrane contains the components required for an ion-selective electrode.

10. Set according to claim 9, wherein the reaction mixture contains 2-ethylhexyl methacrylate (EHMA); 1,6-hexanediol dimethacrylate; AIBN and the components required for an ion-selective electrode.

11. Set according to claim 9, wherein the reaction mixture comprises EHMA, about 0.1 to 5.0% by weight of 1,6-hexandiol dimethacrylate and about 0.1 to 5.0% by weight of free radical initiator.

12. Method for the production of a set according to claim 1, comprising the steps:
providing a common support for at least two ion-selective solid-contact electrodes;
applying for each electrode an inner layer of an electrically conductive polymer to the support;
applying for each electrode an outer ion-selective membrane to the inner layer.

13. Method according to claim 12, wherein the inner layers are cured by heating after applying the electrically conductive polymer to the support.

14. Method according to claim 12, wherein the layers and/or the ion-selective membranes are polymerized in situ.

15. Method for measuring the ion activity in an analysis solution, comprising the steps
immersing a set comprising at least two ion-selective solid-contact electrodes with at least two measuring points which are each selective with respect to different ions of the solution, in an analysis solution,
measuring the potentials between at least two measuring points selective with respect to different ions,
evaluating the potential measurement.

16. Method according to claim 15, wherein the chemical compositions of at least two measuring points differ only through a different ion exchange salt in an ion-selective outer membrane (8).

17. Method according to claim 15, wherein it is a titration or standard addition process.

18. Method according to claim 17, wherein an equivalence point of the titration is determined by registering a change in the potential between two measuring points.

19. Method for the determination of a reference electrode with a potentiometric analysis of a predetermined analysis solution, comprising the steps
defining an ion of the analysis solution, the activity of which does not change or changes in a predictable manner during the analysis,
establishing an ion-selective electrode, which is selective with respect to the defined ion, as the reference electrode.

20. Laboratory apparatus having a set comprising at least two ion-selective solid-contact electrodes according to claim 1, one of the ion-selective solid-contact electrodes serving as a reference electrode.

21. Method for the preparation, of an ion-selective solid-contact electrode wherein the ion-selective solid-contact electrode is sterilized with heat.
